(19) United States
(12) Patent Application Publication
(10) Pub. No.: US 2025/0354865 A1
(43) Pub. Date: Nov. 20, 2025

(54) SENSOR MODULE FOR RAMAN SPECTROSCOPY, ELECTRONIC DEVICE AND METHOD OF CONDUCTING RAMAN SPECTROSCOPY

(71) Applicant: ams International AG, Jona (CH)

(72) Inventors: Remco VERDOOLD, Geldrop (NL); Eva-Maria OELWEINER, Klagenfurt am Wörthersee (AT); Andreas Philipp HASSLER, Klagenfurt am Wörthersee (AT); Thomas HÖPPLER, Jena (DE)

(21) Appl. No.: 18/881,990

(22) PCT Filed: Jun. 28, 2023
(86) PCT No.: PCT/EP2023/067686

§ 371 Date: Jan. 7, 2025

(30) Foreign Application Priority Data
Jul. 8, 2022 (DE) 10 2022 117 049.7

(51) Int. Cl. G01J 3/02; G01J 3/10; G01J 3/28; G01J 3/44

(52) U.S. Cl. CPC G01J 3/0256; G01J 3/0208; G01J 3/10; G01J 3/2803; G01J 3/4412; G01J 2003/2806

(57) ABSTRACT

A sensor module for Raman spectroscopy includes a sensor package which encloses an application specific integrated circuit (ASIC), a light emitter arrangement, a light detector arrangement and a filter arrangement. The light emitter arrangement is electrically connected to the ASIC and operable to emit light with multiple excitation wavelengths to excite Raman scattering in an external probe to be placed outside of the sensor module. The light detector arrangement is operable to generate sensor signals from incident light emitted back from the external probe due to the Raman scattering. The filter arrangement is operable to filter the incident light according to a target passband. The ASIC is operable to drive the light emitter arrangement at the excitation wavelengths to shift a Raman spectral band of the external probe into the passband of the filter arrangement.

This disclosure relates to a sensor module for Raman spectroscopy, an electronic device and a method of conducting Raman spectroscopy.

An object to be achieved is to provide a sensor module for electronic devices that overcomes limitations and provides compact means to conduct Raman spectroscopy in handheld devices. A further object is to provide an electronic device comprising such a sensor module and a method of conducting Raman spectroscopy.

These objectives are achieved with the subject-matter of the independent claims. Further developments and embodiments are described in dependent claims.

In the human body, and most vertebrates, water is used for most of the transportation of supplies and waste products. Additionally, water is used to carefully maintain a constant temperature in the body, using the advantage of the large heat capacity of water. However, a perfect balance, or hydration level, is required for optimal and most efficient performance. A small excess or dehydration does not immediately cause a problem because the body has a large number of redundant systems to counteract problems. However, when over-hydration or dehydration reaches the limit of controllable levels, it becomes dangerous and ultimately can lead to death. Under normal daily circumstances body hydration levels stay well within the save levels. But for those who perform exceptionally well (e.g., athletes or firefighters) or are no longer able to regulate their own hydration level (e.g., the elderly or dialysis patients), measuring hydration levels can become a challenge.

Most of the time body hydration is measured subjectively by performing a skin test. For example, the skin between thumb and index finger is pulled up and released, the time and discoloration could indicate body hydration. However, there are numerous factors influencing the outcome of this test, e.g. skin composition, age and the person performing the test. Alternatively, urine color, with volume, can be measured but vitamin uptake as well as minerals in a diet influences this as well. A third common method is weight measurement, sometimes assisted by body impedance, but as expected, also this measurement is exposed to numerous external factors other than hydration.

One method that is somewhat less subjective is body composition monitoring (e.g., Fresenius body composition monitor). This instrument measures the resistance differences between high and low frequency current. High frequency current is able to easily pass through intra and extra cellular media where low frequency can only pass through extra cellular media. This can provide a more consistent personalized body hydration level and is often used for people in need of dialysis.

Raman spectroscopy is a technique that utilizes monochromatic light (e.g., from a laser) to determine vibrational modes of a molecule. FIG. 6 shows an illustration of the Raman Effect. Briefly, laser light (i.e., photons) with a known and fixed wavelength λₗₐₛₑᵣ (or photon energy) is emitted and strikes a molecule. Due to energy interaction, the molecule can be excited to a higher virtual energy level state. After relaxing back from this state the photon is scattered (emission) with wavelength λ_{scatter}. As illustrated in the energy level scheme on the upper right corner of the drawing, the scattering is considered as Rayleigh type when the energy levels of the laser emitted photon and the scattered photon are equal, i.e. λ_{scatter}=λₗₐₛₑᵣ. Rayleigh scattering is the most observed type of scattering from an illuminated sample. The molecules relax back to their vibrational ground state v=0. However, a smaller fraction of the scattered light will have a different photon energy than the emitted photon energy. This type of inelastic scattering is Raman scattering, where a Stokes shift results in a higher wavelength number and anti-Stokes in a lower wavelength number.

Stokes shifts and anti-Stokes shifts involve higher vibrational states v>0. Thus, the vibration of molecules within a sample will result in a Raman (Stokes and Anti-Stokes) scattering with intensities spread over multiple wavenumbers, which is visualized in the spectrum plot on the bottom left corner of the drawing. As common in Raman spectroscopy, the emitted photon energy is not expressed in wavelength (seen by the spectrometer instrument), but in wavenumbers cm⁻¹, which is the energy shift with respect to the excitation wavelength. Additionally, it is this wavenumber shift, from the excitation wavelength, that is used (Raman shift) for analysis of data. The Raman shift υ̅ derives from

$$\overline{v} = \frac{1}{{λ}_{laser}} - \frac{1}{{λ}_{emission}}$$

wherein λₑₘᵢₛₛᵢₒₙ denotes the emitted Raman peak wavelength, e.g. on a spectrometer.

To date Raman spectrometers often rely on research-grade instrumentation and benchtop systems. Normally, a tuned spectrometer (e.g., a grating with line-pixel sensor) is connected to a lens that receives the Raman scattered light. In the optical path, an optical notch filter is placed that prevents Rayleigh scattering of the laser excitation wavelength λₗₐₛₑᵣ from interfering with the sensor signal or alternatively, a band pass filter is used to stop the excitation light and pass the signal wavelengths of the Raman spectrum. Two very typical Raman spectra are depicted in FIG. 7 with graphs of urea on the left and milk on the right. Urea shows a very strong peak at 1003 cm⁻¹ and milk is a more complex medium with more equal intensity peaks. Visible are peak positions that correspond to present atoms and their bond types. With this molecular fingerprint, it is possible to determine the relative content in complex media.

The capability of Raman scattering of measuring at the molecular selectivity enables to measure levels of individual molecule types in complex media. This enables also to measure the water content in the skin. FIG. 8 shows a typical Raman spectrum of the skin. Depicted is an in-vivo Raman spectrum of the stratum corneum obtained from a human arm. The water protein ratio can be calculated as the ratio between the integrated signal intensities of the gray areas. Experimental conditions include a signal collection time of 3 s, laser power at 100 mW, and excitation wavelength of 720 nm.

There are two main peaks in the Raman spectrum, one for solids of cells, like proteins and lipids and the second for water content of the cells and extracellular media. The solid content will remain mostly stable at various levels of water content. The spectral graph illustrates the variation in hydration level, by change in peak intensity for the water Raman band. For the actual measurement of water content in skin it is not required to have absolute Raman spectral information but a ratio of the two highlighted bands would be sufficient. This enables using only two spectral filters. One that gives the signal intensity for proteins and lipids, and a second filter that gives the signal intensity for water. The ratio between those two numbers is an indication for hydration. It does require a normality calibration to obtain a normal hydration level ratio. However, this can also be obtained from trend information during multi day measurements.

The level of hydration or water in the skin varies with the depth from the skin surface (see Thesis—Peter Caspers, in-vivo skin characterization by confocal Raman micro spectroscopy, 2003, university of Rotterdam, isbn 978-90-6734-366-4 and Peter J. Caspers, Gerald W. Lucassen, Elizabeth A. Carter, Hajo A. Bruining, and Gerwin J. Puppels, (2001), In Vivo Confocal Raman Microspectroscopy of the Skin: Noninvasive Determination of Molecular Concentration Profiles, 2001, VOL. 116, NO. 3 Mar. 2001, IN VIVO RAMAN SPECTROSCOPY OF SKIN by The Society for Investigative Dermatology, Inc.). At a depth of 200 μm from the skin surface the hydration level is approximately 65% as illustrated in FIG. 9. FIG. 9 shows in-vivo water concentration profiles of the stratum corneum, as found in the citations above. The graph on the left shows four water concentration profiles, calculated from Raman measurements on the volar aspect of the forearm. Different symbols were used for profiles obtained for different measurement locations. The graph on the right shows four water concentration profiles based on Raman measurements on the thenar. Different symbols demarcate different measurement locations. The left hand ordinate is the ratio between the Raman signal intensities of water and protein. The right hand ordinate represents the calculated absolute water content in mass-% (grams of water per 100 g of wet tissue). Note that this is a non-linear scale.

In another application, Raman spectroscopy can be used to conduct measurements of pathogens such as bacteria. Every organism will have different ratios of chemicals and molecules present in their body. The simpler the organism the less complex the Raman spectrum would look like. For example, in bacteria there are various classifications. At first there is GRAM positive or negative, this is an indicator for the construction of the cell membrane as shown in FIG. 10. Here, it is possible to see that different molecules are present and that this will influence the Raman response from these types of organisms. FIG. 10 shows Gram positive vs Gram negative on the left. On the right Raman SERS spectrum of two Gram positive and two Gram negative bacteria. Next to the membrane structure of bacteria their content like proteins and amino acids will add to the complexity of the Raman spectrum. Using a Cluster analysis one can separate the complexity between samples as shown in FIG. 11. Figure shows 11 shows a cluster analysis of Gram positive and Gram negative bacteria.

Lately there has been a tremendous advancement in the field of photonics. Thanks to photonics Raman spectroscopy has become much more accessible to users in all fields and provides more objective means for various aspects of applications, such as Biosensing. Footprints of complete spectrometers can be further miniaturized with the help of ever more compact integrated optics, optoelectronics and laser sources. However, Raman spectrometers still remain research-grade instrumentation and benchtop systems, despite the growing need for mobile devices such as mature spectrometers in Smart Watches, medical and point-of-care or other handheld devices. Biosensing applications to detect skin constituents like urea, lactate and interstitial fluid or blood glucose are only one example, measurements of pathogens (e.g. bacteria) another. Compact handheld devices are expected to have a huge impact on most potential spectrometer applications, e.g. material analysis, environmental analysis, biosensors, Smart Health, Medsumer, point-of-care, medical etc.

The following relates to an improved concept in the field of Raman spectroscopy. One aspect relates to a sensor module for Raman spectroscopy, i.e. a miniaturized measurement system to conduct Raman spectroscopy. Possible applications comprise a fingerprinting measurement system of bacteria or viruses. This system can be used for many other measurement applications as well, such as ripeness of fruits, quality of foods, raw material safety etc. Another application includes skin hydration, e.g. by measuring the ratio between solids and water content of the skin using a single wavelength band filter to detect the Raman peaks.

Further aspects relate to a combination of a spectral sensor with a number of diodes, integrated in an ASIC, or a number of diodes with spectral filters readout by an ASIC, in combination with only one or a few narrowband light emitter(s) and a miniature housing, all fitting in a volume of smaller than 2 cm³. Potentially, the light emitter can be modulated, which modulation frequency may be seen back in the intensity modulation of the detected Raman signal. Additionally, multiple excitation sources can be used to spread the power density over the skin and stay below safety limits for skin illumination. Additionally, different lenses can have different focus depths to create a depth profile of the skin, with respect to water or any other Raman active material of the skin or tissue.

In at least one embodiment, a sensor module for Raman spectroscopy comprises a sensor package. The sensor package encloses an application specific integrated circuit, or ASIC for short, a light emitter arrangement, a light detector arrangement and a filter arrangement.

The light emitter arrangement, e.g. a semiconductor light emitter arrangement, is electrically connected to the ASIC and operable to emit light with multiple excitation wavelengths to excite Raman scattering in an external probe to be placed outside of the sensor module. The light detector arrangement is operable to generate sensor signals from incident light emitted back from the external probe due to the Raman scattering. The filter arrangement is operable to filter the incident light according to a target passband. The ASIC is operable to drive the light detector arrangement at the excitation wavelengths to shift a Raman spectral band of the external probe into the passband of the filter arrangement.

The shifting of Raman spectral band allows to probe sections of a Raman spectrum with just a single filter or a set of filters to extend the range of sections which can be probed. This way, the sensor module eliminates the use of a high resolution spectrometer and complex optics and replaces this by a number of excitation wavelengths and set of well-defined passband filters. This greatly simplifies the measurement system and reduces cost prices from tens of thousands to tens of dollars. As a consequence, Raman spectroscopy may be conducted in handheld devices.

In at least one embodiment, the light emitter arrangement comprises a single light emitter operable to emit light with multiple emission lines according to the multiple excitation wavelengths. A single light emitter may suffice to provide multiple excitation wavelengths, which renders the sensor module more compact. Said light emitter may emit discrete multiple excitation wavelengths all at once, so that individual lines may need to be filtered out.

In at least one embodiment, the light emitter arrangement comprises a single tuneable light emitter operable to emit light with tuneable emission lines according to the multiple excitation wavelengths. A single light emitter may suffice to provide multiple excitation wavelengths, which renders the sensor module more compact. Said tuneable light emitter may emit a single discrete excitation wavelength at a time and be tuned to emit another. This way there may be no need to filter out individual lines.

In at least one embodiment, the light emitter arrangement comprises an array of light emitters with different emission lines. The light emitters of the array are operable to emit light with at least one emission line according to the multiple excitation wavelengths. An array of light emitters may require more space but could be manufactured more easily by means of CMOS technology, for example. Instead of tuning, multiple excitation wavelengths may readily be available, e.g. under control of the ASIC.

In at least one embodiment, the light emitter arrangement comprises at least one of a laser diode or a laser surface emitter, e.g. a VCSEL, as light emitter.

In at least one embodiment, the light detector arrangement comprises a single light detector and/or a single semiconductor light detector. The filter arrangement comprises a filter which is arranged in front of the light detector. A single light detector may suffice as spectral features of the probe are shifted into the passband of the filter or passbands of filters due to the multiple excitation wavelengths, which renders the sensor module more compact.

In at least one embodiment, the light detector arrangement comprises an array of light detectors and/or an array of semiconductor light detectors. The filter arrangement comprises multiple filters arranged in front of the light detectors, respectively. The array may be arranged with a single filter to increase signal-to-noise ratio, for example. Instead there may be a dedicated filter in front of respective light detectors in order to be able to probe more sections or spectral features of an external probe. Furthermore, using an array also facilitates use of the sensor module for more external probes, as the target passbands can be adjusted to cover more spectral features.

In at least one embodiment, the filter in front of the single light detector comprises a broad passband, or broadband-filter for short. Alternatively, multiple filters arranged in front of the light detectors comprise narrow passbands, or narrowband-filters for short. Bandwidth may account for the expected width of spectral features, i.e. peaks or bands, in the desired Raman spectra.

In at least one embodiment, the narrowband-filters have non-overlapping passbands with discrete center wavelengths. Non-overlap may further spread the spectral range that can be probed.

In at least one embodiment, the light detector arrangement comprises at least one of a photon counter, e.g. a single photon avalanche diode, or SPAD, an avalanche photo diode, or APD, a silicon photomultiplier, or SiPM, a photodiode or a charge coupled device, or CCD, or a MEMS photo multiplier, or PM, as light detector.

In at least one embodiment, the ASIC further comprises a modulator and a lock-in amplifier. The modulator is operable to provide an AC drive signal with a modulation frequency and to provide a reference signal associated with the AC drive signal. Furthermore, the lock-in amplifier is operable to receive the sensor signals and to receive the reference signal from the modulator, and to perform phase-locked detection of the modulation frequency in the sensor signals to determine a phase and an amplitude from the sensor signals using the reference signal. Modulated excitation enables to do phase-locked detection of the modulation frequency in the Raman sensor signal, which further improves the sensitivity and accuracy of the detection.

In at least one embodiment, the sensor package further encloses a lens arrangement. The lens arrangement is arranged to direct the emitted light to the external probe to excite Raman scattering. In addition, or alternatively, the lens arrangement is arranged to direct the incident light to the light detector arrangement. The lens arrangement may increase signal-to-noise ratio by way of focusing in both excitation and detection. Furthermore, the lens arrangement may provide different focal lengths in order to excite the external probe with different penetration depths.

In at least one embodiment, the lens arrangement is further operable to direct the emitted light to the external probe under an angle different from normal incidence so as to provide angled illumination with an angled illumination source which helps to reduce the impact of auto fluorescence from the target.

In at least one embodiment, an electronic device comprises a sensor module for Raman spectroscopy according to one of the aforementioned aspects. The electronic device comprises a host system and the sensor module is embedded in and electrically connected to the host system. The host system comprises one of a mobile device, smartphone, handheld computer, Smart Watch, medical device, point-of-care device.

Furthermore, a method of conducting Raman spectroscopy is suggested. The method is conducted using a sensor module comprising a sensor package which encloses an application specific integrated circuit, or ASIC, a light emitter arrangement electrically connected to the ASIC, a light detector arrangement and a filter arrangement. The method comprises the step of emitting light with multiple excitation wavelengths to excite Raman scattering in an external probe to be placed outside of the sensor module. Another steps involves generating sensor signals from incident light emitted back from the external probe due to the Raman scattering. Another steps involves filtering the incident light according to a target passband. Another steps involves, using the ASIC, driving the light detector arrangement at the excitation wavelengths to shift a Raman spectral band of the external probe into the passband of the filter arrangement.

Further embodiments of the method become apparent to the skilled reader from the aforementioned embodiments of the sensor module and of the electronic device, and vice-versa.

The proposed concept may have one or more of the following advantages:
less components needed,
offers more integrated solution, e.g. less parasitics enables to better measure the weak Raman signals and application of modulated excitation may enable to do phase-locked detection of the modulation frequency in the Raman signal, which further improves the sensitivity and accuracy of the detection,
allows to implement true point-of-care device. Currently, spectroscopy measurements are almost exclusively performed at the clinical laboratory,
possibility for point-of-care measurement with disposable components,
performance, low cost, low COGS,
possibility to implement in wearable sensor host systems,
potential to have absolute measurements of body hydration on the measurement location,
an excitation from multiple sources may decrease the illumination density at the skin entrance, well within safety limits, while keeping sufficient intensity at the focal point for the Raman Signal generation and different lenses on each excitation source can have variable focus depth, through which a depth curve can be measured.
sensor module can be as small as volume <2 cm³

### BRIEF DESCRIPTION OF THE DRAWINGS

The following description of figures may further illustrate and explain aspects of the sensor module for Raman spectroscopy, electronic device and the method of conducting Raman spectroscopy. Components and parts of the sensor module that are functionally identical or have an identical effect are denoted by identical reference symbols. Identical or effectively identical components and parts might be described only with respect to the figures where they occur first. Their description is not necessarily repeated in successive figures.

In the figures:

FIG. 1 shows a first exemplary embodiment of a sensor module for Raman spectroscopy,

FIG. 2 shows Raman spectra with an example set of spectral bands and excitation wavelengths for the first exemplary embodiment,

FIG. 3 shows a second exemplary embodiment of a sensor module for Raman spectroscopy,

FIG. 4 shows a third exemplary embodiment of a sensor module for Raman spectroscopy,

FIG. 5 shows Raman spectra with an example set of spectral bands and excitation wavelengths for the third exemplary embodiment,

FIG. 6 shows an illustration of the Raman Effect,

FIG. 7 shows Raman spectra of urea and milk,

FIG. 8 shows an in-vivo Raman spectrum of the stratum corneum obtained from a human arm,

FIG. 9 shows in-vivo water concentration profiles of the stratum corneum,

FIG. 10 shows an illustration of Gram positive vs Gram negative bacteria, and

FIG. 11 shows a cluster analysis of Gram positive and Gram negative bacteria.

### DETAILED DESCRIPTION

In the following several exemplary embodiments of a sensor module for Raman spectroscopy are presented. The sensor module comprises a sensor package which encloses the components of the module. For example, the sensor package comprises a molded housing (not shown) to mount or place the components into. The sensor module encloses components including an application specific integrated circuit 10, or ASIC for short, a semiconductor light emitter arrangement 20, a semiconductor light detector arrangement 30 and a filter arrangement 40. In the examples discussed below a lens arrangement is also arranged into the sensor package. Alternatively, a non-semiconductor light emitter arrangement and/or light detector arrangement can be implemented.

For example, the molded housing comprises a hollow molded body which is mounted on and connected to the ASIC 10, e.g. by means of a carrier. Furthermore, the semiconductor light emitter arrangement 20 and the semiconductor light detector arrangement 30 are placed behind respective apertures to emit light out of the sensor module and receive incident light. The housing can be arranged with chambers, one chamber for the semiconductor light emitter arrangement and another chamber for the semiconductor light detector arrangement. The semiconductor light emitter arrangement and the semiconductor light detector arrangement can be optically isolated by means of a light barrier, e.g. a wall in the housing separating the chambers.

The semiconductor light detector arrangement can be integrated into the ASIC 10, or, together with the ASIC, form an integrated circuit, such as a CMOS integrated circuit, mounted on a common substrate or carrier. The semiconductor light emitter arrangement 20 can either be integrated into the ASIC or the integrated circuit or be electrically connected to the integrated circuit or ASIC as external components.

The semiconductor light emitter arrangement 20 comprises one or more light emitters 21, such as semiconductor laser diodes and/or resonant cavity light emitting devices. These devices feature coherent emission to generate light at various excitation wavelengths. A resonant cavity light emitting device can be considered a semiconductor device which is operable to emit coherent light based on a resonance process. In this process, the resonant cavity light emitting device may directly convert electrical energy into light, e.g. when pumped directly with an electrical current to create amplified spontaneous emission. However, instead of producing stimulated emission only spontaneous emission may result, e.g. spontaneous emission perpendicular to a surface of the semiconductor is amplified.

One example relates to vertical cavity surface emitting laser, VCSEL, diodes. VCSELs are an example of a resonant cavity light emitting device and feature a beam emission that is perpendicular to a main extension plane of a top surface of the VCSEL. The VCSEL diode can be formed from semiconductor layers on a substrate, wherein the semiconductor layers comprise distributed Bragg reflectors enclosing active region layers in between and thus forming a cavity. VCSELs and their principle of operation are a well-known concept and are not further detailed throughout this disclosure. For example, the VCSEL diode is configured to have an emission wavelength of 940 nm, 850 nm, or another natural wavelength. Other emission wavelengths include 660 nm, 671 nm, 680 nm, and 785 nm. However, VCSELs can also be tuneable and driven by the ASIC to emit at various wavelengths. The VCSEL diode can be configured to emit coherent laser light when forward biased, for instance.

The semiconductor light detector arrangement comprises one or more light detectors 31, such as photon counters, e.g. single photon avalanche diodes, or SPADs, avalanche photo diodes, or APDs, silicon photomultipliers, or SiPMs, semiconductor photodiodes or charge coupled devices, or CCDs, or MEMS photo multipliers, or PMs.

The light detectors 31 are complemented with the filter arrangement 40, which filters incident light according to one or more target passbands. The filter arrangement comprises one or more optical filters 41, such as interference filters or dichroic filters, Plasmon filters and/or absorption filters, or a combination thereof. The filters can be arranged in a filter layer with one or more sections dedicated to a respective light detector. The filters may also be placed on or integrated into a corresponding light detector.

The target passband of a filter 41 can be a broad passband, i.e. the filter is a broadband-filter, or narrow passbands, i.e. the filter is a narrowband-filter. Broad is considered 50 nm and larger and narrow is considered smaller than 10 nm, for example. The term “target” is used to indicate that the passband is chosen with a desired target or probe in mind. For example, a substance to be measured is known to feature a Raman spectral band in a defined spectral range when excited with a given excitation wavelength. Then the target passband can be chosen to cover (or pass) said Raman spectral band. This way the passband may cover a characteristic spectral feature of a probe and, in turn, it may suffice that only said spectral band is probed to detect the desired target, or substance.

Furthermore, the sensor package comprises a lens arrangement 50 with lenses 51, e.g. micro-lenses, placed on or integrated directly into a corresponding light detector 31. The lens arrangement directs emitted light to an external probe 60 to excite Raman scattering. Furthermore, the lens arrangement may also be used to direct the incident light to the light detector arrangement. For example, lenses can have different focal lengths to focus emitted light into different depths of the external target. Moreover, lenses may be tilted in the sensor package so as to direct the emitted light to the external probe under an angle different from normal incidence. This way, angled illumination of the external target is possible with an angled illumination source which helps to reduce the impact of auto fluorescence from the target. Furthermore, additional lenses may be provided to focus incident light onto the respective light detectors to increase signal-to-noise levels.

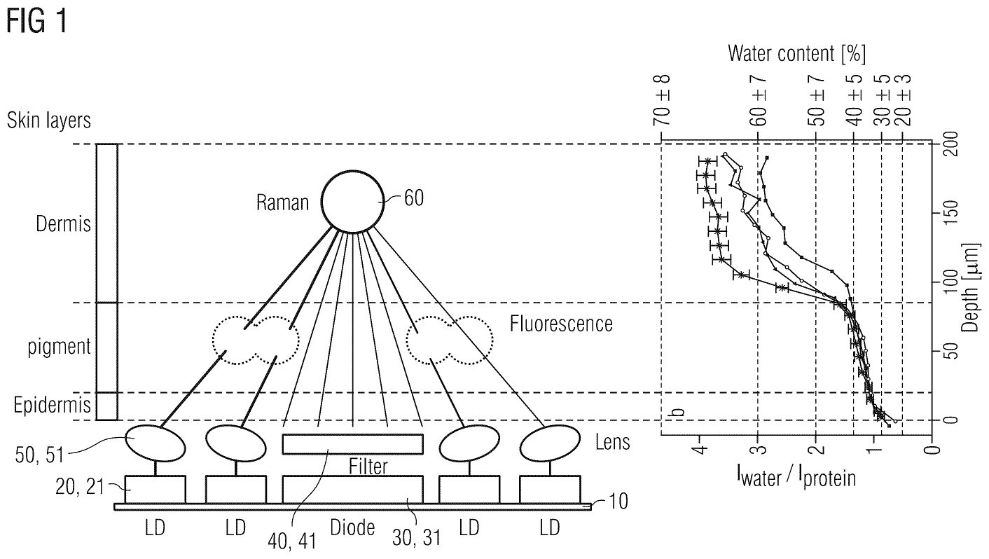

FIG. 1 shows a first exemplary embodiment of a sensor module for Raman spectroscopy. In this embodiment the filter arrangement 40 comprises only a single optical filter 41, and a high performance light detector 31 forms the semiconductor light detector arrangement 30 on the sensing side. Alternatively, more than a single light detector can form the semiconductor light detector arrangement and be placed behind the same single optical filter 41 to further increase signal-to-noise ratio. On the source side the semiconductor light emitter arrangement 20 is formed by four different light emitters 21, for example, such as laser diodes, each operable to emit with a unique excitation wavelength. This greatly simplifies the measurement system. Furthermore, this embodiment employs tilted lenses which form the lens arrangement in front of the light emitters.

In operation, light is excited by the light emitters 21 and focused to a specific depths at the external target, e.g. at 80 to 500 μm into the dermis of human tissue. Other depths are possible as shown by the depth-water graph on the right hand side of the drawing. Raman emission from molecules is scattered and received by the light detector 31. Due to the filter 41 only light with wavelengths defined by the target passband pass and are incident on the light detector to generate respective sensor signals. In this example, the filter accepts energy levels for solids or water.

The sensor signals can be analyzed, e.g. by the ASIC 10 or by an external processing unit (not shown). For example, a ratio of sensor signals (or channels) collected for different excitation wavelengths can give an indication of the presence of a given spectral feature, e.g. a peak or band, of the external target 60. For example, a ratio of sensor signals related to protein and water may give an indication of body hydration. The melanin in skin will result in auto fluorescence, therefore the angle of illumination could differ from the position of reception.

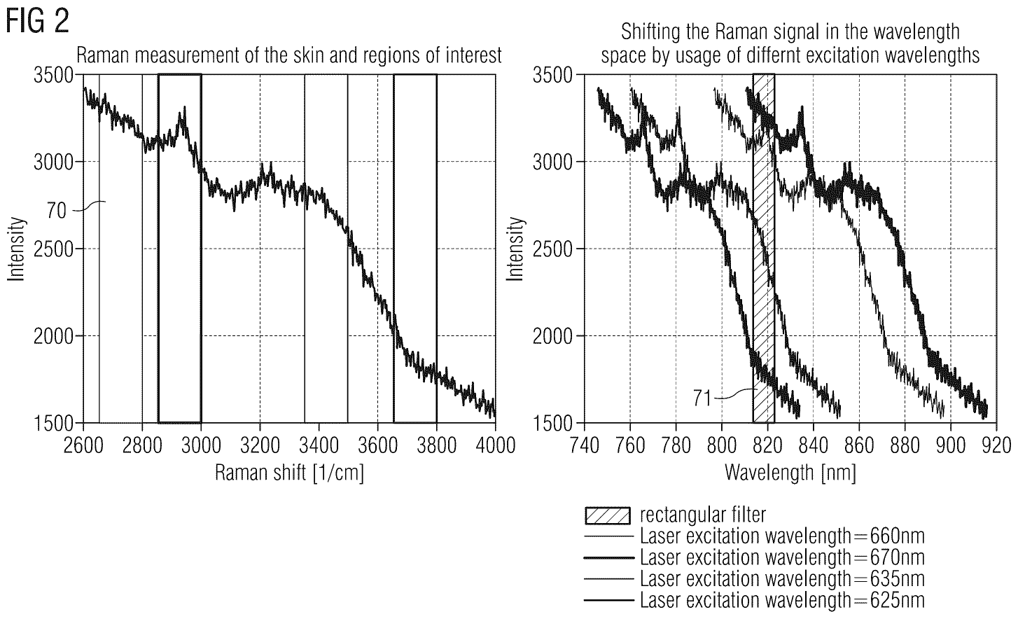

FIG. 2 shows Raman spectra with an example set of spectral bands and excitation wavelengths for the first exemplary embodiment. The graph on the left shows a Raman spectrum of a human skin, intensity in arbitrary units vs. Raman shift in wavenumber cm⁻¹. The graph further indicates four spectral bands 70, or regions of interest. These regions of interest are related to the four excitation wavelengths which can be provided by the light emitters. In this example, a first excitation wavelength is 660 nm, a second excitation wavelength is 670 nm, a third excitation wavelength is 635 nm, and a fourth excitation wavelength is 625 nm.

The graph on the right represents intensity in arbitrary units vs. wavelength in nm. The rectangular area represents the target passband 71 of the optical filter 41. Furthermore, four Raman spectra are depicted, resulting from excitation with the four excitation wavelengths, respectively. As a consequence of the different excitation, the Raman spectra are shifted with respect to wavelength space. For example, a peak in the Raman spectrum is shifted into the target passband using the first excitation wavelength at 660 nm. In other words, changing the excitation wavelengths shifts different Raman spectral bands of the external probe (here human skin) into the passband of the filter. This way, the external target 60 can be probed in sections, or regions of interest.

The measurements performed with a desktop Raman spectrometer, however, involve computing or probing the complete scattering spectrum of the Raman signal. However, as the measurement discussed in FIG. 1 uses different excitation wavelengths, merely part of the Raman spectrum can be probed, e.g. regions of interest, which include a desired spectral feature, e.g. a peak or band of a known specimen or substance. For example, for detecting the Raman signal peaks of solids and water, by way of shifting the spectra into a single target passband 71 a single filtered sensor could be sufficient.

It should be noted that hydration is not the only potential use case of this device. Equally for other compounds more than two specific wavelengths can be selected and detected by dedicated light detectors with optical filters 41 on top, either separated or integrated in a spectra sensor configuration, embedded in a readout ASIC 10.

An advantage of using a single spectral filter 41 over multiple filters is that the light detector(s) 31 can have a large surface area, and optimize the angle of incidence of the lenses. Also the filter fabrication step is greatly simplified and will have reduced stresses in the material because only a single filter is present on the carrier medium (e.g. glass). Additionally, the light emitters 21 can be all placed around the sensor or a single laser can be tuned to various wavelengths as will be discussed next.

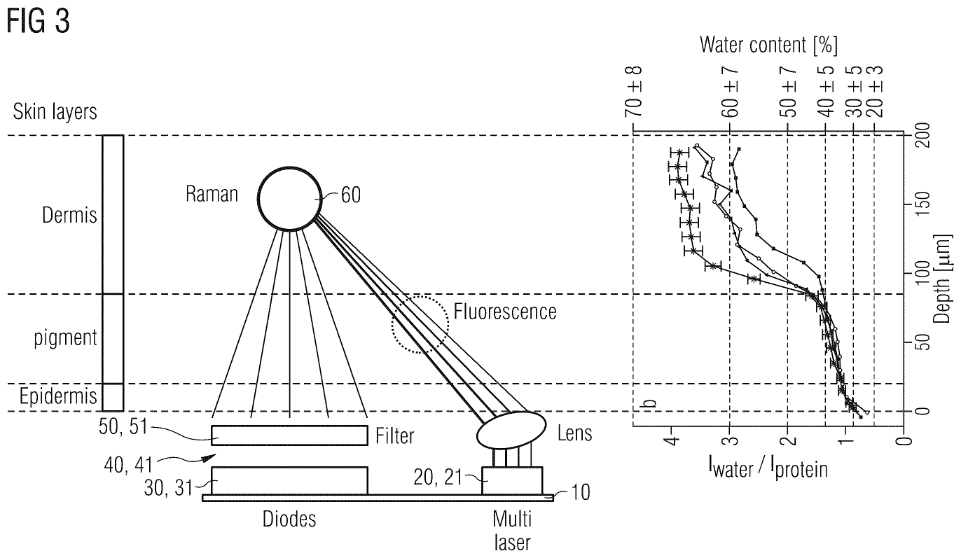

FIG. 3 shows a second exemplary embodiment of a sensor module for Raman spectroscopy. This example is closely related to the one discussed in respect of FIG. 1. Instead of four single light emitters 21, however, a single light emitter is used. This single light emitter is operable to emit light with four different excitation wavelengths, e.g. the first to fourth excitation wavelengths introduced above. In general, it is possible to have the excitation wavelengths realized as distinct emission lines or instead the light emitter is tuneable to provide emission lines from a continuous spectral range. This further simplifies the measurement system, e.g. in terms of space requirement and compactness. For example, semiconductor laser diodes such as VCSELs can be designed with tuneable emission.

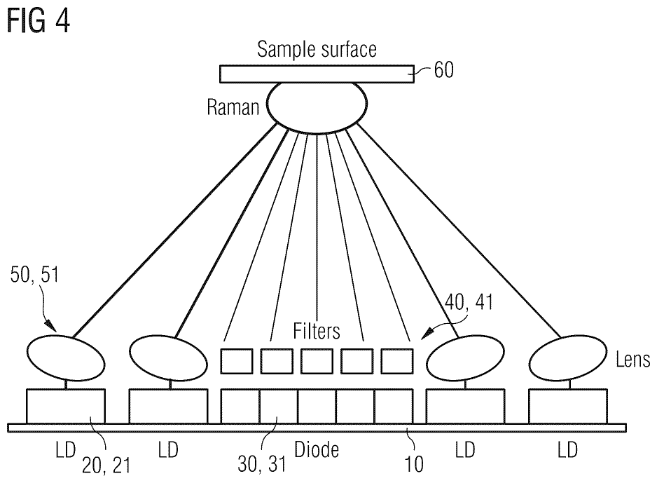

FIG. 4 shows a third exemplary embodiment of a sensor module for Raman spectroscopy. In this embodiment the filter arrangement 40 comprises several optical filters 41 with different target passbands 71 (here five as an example), and an array of respective (five) light detectors 31 forms the semiconductor light detector arrangement 30 on the sensing side. On the source side the semiconductor light emitter arrangement 20 is formed by four different light emitters 21, such as laser diodes, each operable to emit with a unique excitation wavelength. This greatly simplifies the measurement system. Furthermore, this embodiment employs tilted lenses which form the lens arrangement in front of the light emitters.

Operation of the sensor module is as discussed above. A difference involves that the Raman spectra can be shifted according to the excitation wavelengths into several target passbands 71, as opposed to a single passband as discussed above. For example, using five spectral band filters with respective non-overlapping target passbands, numerous spectral features, e.g. peaks and bands of interest, can be measured. By using three excitation wavelengths it is possible to measure three Raman bands with a single filter. So in general with five filters and four light emitters a total of 20 spectral bands can be measured.

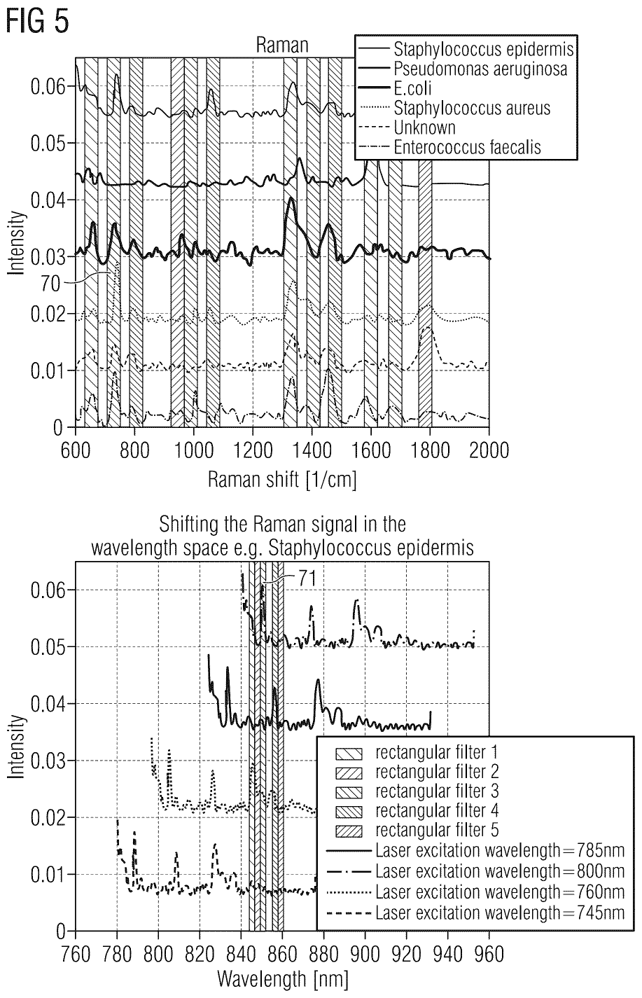

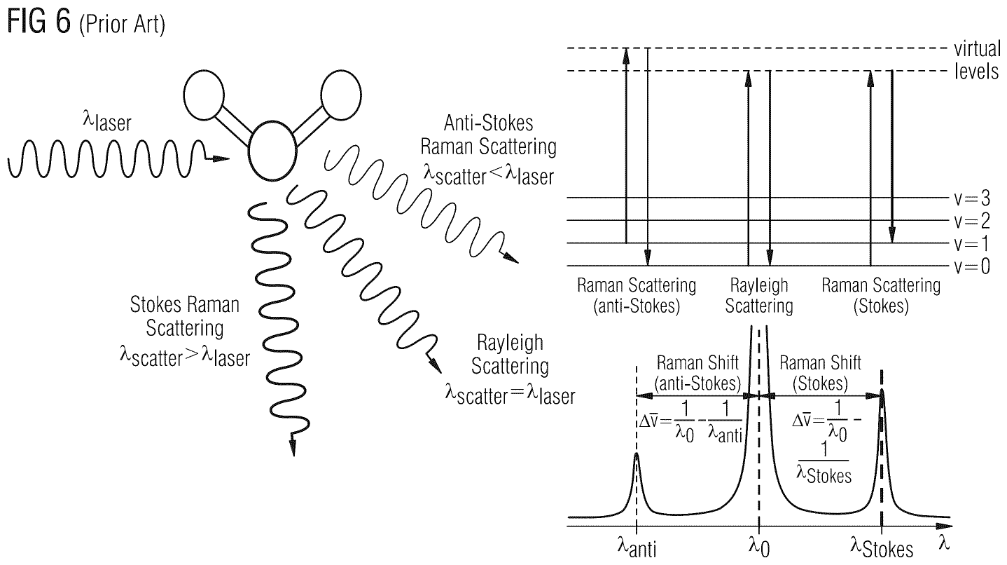

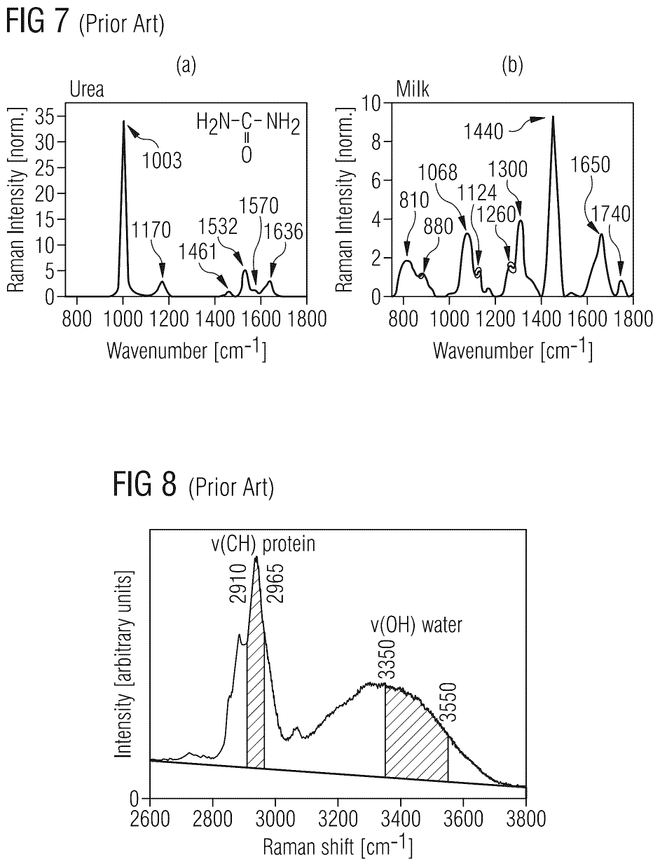

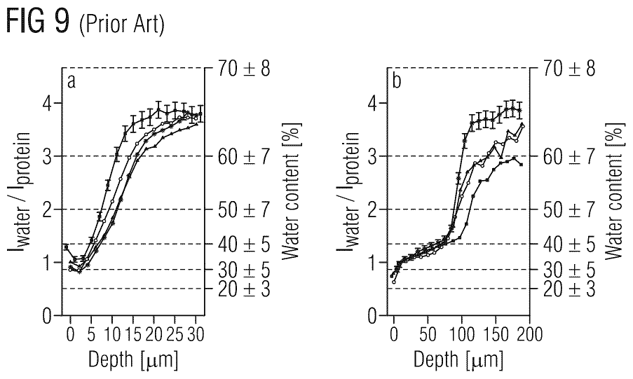

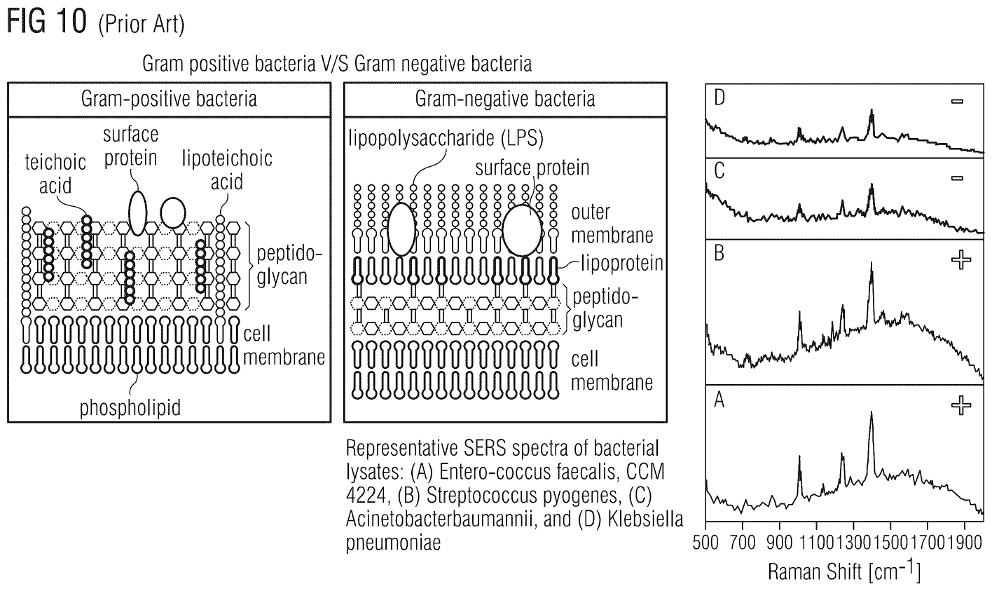

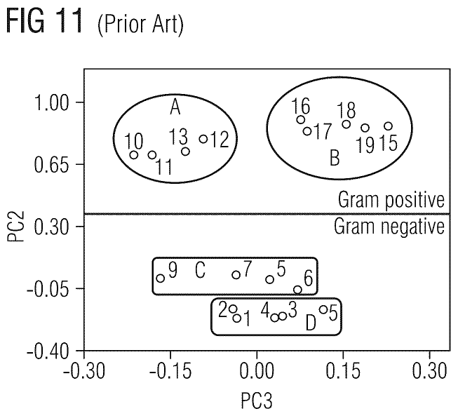

FIG. 5 shows Raman spectra with an example set of spectral bands and excitation wavelengths for the third exemplary embodiment. The graph on the left shows Raman spectra of various bacteria, intensity in arbitrary units vs. Raman shift in wavenumber cm⁻¹. The graph further indicates several bands or regions of interest. These regions of interest are related to the excitation wavelengths which can be provided by the light emitters. In this example, a first excitation wavelength is 785 nm, a second excitation wavelength is 800 nm, a third excitation wavelength is 760 nm, and a fourth excitation wavelength is 745 nm.

The graph on the right represents intensity in arbitrary units vs. wavelength in nm. The rectangular areas represent the target passbands 71 of the optical filters 41. Four Raman spectra of Staphylococcus epidermis are depicted, resulting from excitation with the four different excitation wavelengths, respectively. As a consequence of the different excitation, the Raman spectra are shifted with respect to wavelength space. For example, different peaks of the Raman spectrum are shifted into the target passbands 71 using the different excitation wavelengths. In other words, changing the excitation wavelengths shifts different Raman spectral bands of the external probe 60 (here Staphylococcus epidermis) into the passbands 71 of the filters 41. This way, the external target can be probed in sections, or regions of interest.

The measurements performed with a desktop Raman spectrometer cover the complete scattering spectrum of the Raman signal. However, as the measurement discussed in FIG. 4 uses different excitation wavelengths, merely parts of the Raman spectrum can be probed, e.g. regions of interest, which include desired spectral features, e.g. a peak or band of a known specimen or substance. For example, for detecting the Raman signal peaks of bacteria, by way of shifting the spectra a target passbands and/or a filtered sensor could be sufficient.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

Furthermore, as used herein, the term “comprising” does not exclude other elements. In addition, as used herein, the article “a” is intended to include one or more than one component or element, and is not limited to be construed as meaning only one.

This patent application claims the priority of German patent application 102022117049.7, the disclosure content of which is hereby incorporated by reference.

### REFERENCES

10 application specific integrated circuit, ASIC
20 light emitter arrangement
21 light emitter
30 light detector arrangement
31 light detector
40 filter arrangement
41 filter
50 lens arrangement
51 lens
60 external probe
70 spectral band
71 target passband

1. A sensor module for Raman spectroscopy, comprising a sensor package which encloses:
an application specific integrated circuit (ASIC),
a light emitter arrangement electrically connected to the ASIC and operable to emit light with multiple excitation wavelengths to excite Raman scattering in an external probe to be placed outside of the sensor module,
a light detector arrangement operable to generate sensor signals from incident light emitted back from the external probe due to the Raman scattering, and
a filter arrangement operable to filter the incident light according to a target passband; and wherein:
the ASIC is operable to drive the light emitter arrangement at the excitation wavelengths to shift a Raman spectral band of the external probe into the passband of the filter arrangement.

2. The sensor module according to claim 1, wherein the light emitter arrangement comprises a single light emitter operable to emit light with multiple emission lines according to the multiple excitation wavelengths.

3. The sensor module according to claim 1, wherein the light emitter arrangement comprises a single tuneable light emitter operable to emit light with tuneable emission lines according to the multiple excitation wavelengths.

4. The sensor module according to claim 1, wherein
the light emitter arrangement comprises an array of light emitters with different emission lines, and
the light emitters of the array are operable to emit light with at least one emission line according to the multiple excitation wavelengths.

5. The sensor module according to claim 1, wherein the light emitter arrangement comprises at least one of a laser diode or a laser surface emitter, e.g. a VCSEL, as light emitter.

6. The sensor module according to claim 1, wherein
the light detector arrangement comprises a single light detector, and
the filter arrangement comprises a filter which is arranged in front of the light detector.

7. The sensor module according to claim 1, wherein
the light detector arrangement comprises an array of light detectors, and
the filter arrangement comprises multiple filters arranged in front of the light detectors, respectively.

8. The sensor module according to claim 6, wherein:
the filter in front of the single light detector comprises a broad passband, or broadband-filter, or
multiple filters arranged in front of the light detectors comprise narrow passbands, or narrowband-filters.

9. The sensor module according to claim 8, wherein the narrowband-filters have non-overlapping passbands with discrete center wavelengths.

10. The sensor module according to claim 1, wherein the light detector arrangement comprises at least one of a photon counter, e.g. a single photon avalanche diode, or SPAD, an avalanche photo diode, or APD, a silicon photomultiplier, or SiPM, a photodiode or a charge coupled device, or CCD, or a MEMS photo multiplier, or PM, as light detector.

11. The sensor module according to claim 1, wherein the ASIC further comprises:
a modulator operable to provide an AC drive signal with a modulation frequency and to provide a reference signal associated with the AC drive signal,
a lock-in amplifier operable to receive the sensor signals and to receive the reference signal from the modulator, and to perform phase-locked detection of the modulation frequency in the sensor signals to determine a phase and an amplitude from the sensor signals using the reference signal.

12. The sensor module according to claim 1, wherein
the sensor package further encloses a lens arrangement, and
the lens arrangement is arranged to direct the emitted light to the external probe to excite Raman scattering, and/or
the lens arrangement is arranged to direct the incident light to the light detector arrangement.

13. The sensor module according to claim 12, wherein the lens arrangement is further operable to direct the emitted light to the external probe under an angle different from normal incidence so as to provide angled illumination.

14. The sensor module according to claim 1, wherein the light detector arrangement is integrated into the ASIC and/or the semiconductor light emitter arrangement is integrated into the ASIC.

15. An electronic device, comprising:
a sensor module for Raman spectroscopy according to claim 1, and
a host system comprising one of a mobile device, smartphone, handheld computer, Smart Watch, medical device, point-of-care device.

16. A method of conducting Raman spectroscopy with a sensor module for Raman spectroscopy, the sensor module comprising a sensor package which encloses:
an application specific integrated circuit (ASIC),
a light emitter arrangement electrically connected to the ASIC,
a light detector arrangement,
a filter arrangement;
the method comprising the steps of:
emitting light with multiple excitation wavelengths to excite Raman scattering in an external probe to be placed outside of the sensor module,
generating sensor signals from incident light emitted back from the external probe due to the Raman scattering,
filtering the incident light according to a target passband, and
using the ASIC, driving the light detector arrangement at the excitation wavelengths to shift a Raman spectral band of the external probe into the passband of the filter arrangement.

17. (canceled)
